(19) United States
(12) Patent Application Publication
(10) Pub. No.: US 2019/0290868 A1
(43) Pub. Date: Sep. 26, 2019

(54) PORTABLE ANESTHESIA APPARATUS AND TRANSPORTATION CART

(71) Applicant: Jarom C. HEATON, Round Rock, TX (US)

(72) Inventor: Jarom C. HEATON, Round Rock, TX (US)

(21) Appl. No.: 16/363,615

(22) Filed: Mar. 25, 2019

Related U.S. Application Data
(60) Provisional application No. 62/647,977, filed on Mar. 26, 2018.

(51) Int. Cl. A61M 16/01; A61B 50/13; A61M 16/18; A61M 16/22

(52) U.S. Cl. CPC A61M 16/01; A61B 50/13; A61M 16/1005; A61M 16/22; A61M 16/186

(57) ABSTRACT

A portable anesthesia apparatus may include an anesthesia transportation cart, a hospital-grade anesthesia machine mounted on the anesthesia transportation cart, and a hospital-grade CO₂ absorber mounted on the anesthesia transportation cart. The portable anesthesia apparatus may have an apparatus footprint area A_{FA} of not greater than about 1.0 m².

### CROSS-REFERENCE TO RELATED APPLICATION(S)

This application claims priority under 35 U.S.C. § 119(e) to U.S. Provisional Patent Application No. 62/647,977, entitled “PORTABLE ANESTHESIA APPARATUS AND TRANSPORTATION CART,” by Jarom C. HEATON, filed on Mar. 26, 2018, and said provisional application is incorporated by reference herein in its entirety for all purposes.

### BACKGROUND

### Field of the Disclosure

The following is directed to a portable anesthesia apparatus and a portable anesthesia apparatus transportation cart.

### Description of the Related Art

Anesthesia equipment and transportation carts for various components necessary for the application of anesthesia are typically used at a single worksite (i.e. a hospital, a dental office, etc.) and are normally transported on a component by component basis. In other words, individual anesthesia equipment components are contained or attached to separate transportation carts and moved individually throughout the worksite. Alternatively, multiple components are contained and arranged on transportation carts that occupy relatively large amounts of space in order to allow convenient access to each component. However, due to the growth of mobile, onsite anesthesia service providers that are certified to perform and offer full service application of anesthesia, the industry continues to demand improved options for transporting anesthesia equipment between multiple locations in a functional and convenient manner.

### SUMMARY

According to a first aspect, a portable anesthesia apparatus may include an anesthesia transportation cart, a hospital-grade anesthesia machine mounted on the anesthesia transportation cart, and a hospital-grade CO₂ absorber mounted on the anesthesia transportation cart. The portable anesthesia apparatus may have an A_{FA} of not greater than about 1.0 m².

According to another aspect, a portable anesthesia apparatus may include an anesthesia transportation cart, a hospital-grade anesthesia machine mounted on the anesthesia transportation cart, and a hospital-grade CO₂ absorber mounted on the anesthesia transportation cart. The portable anesthesia apparatus may have an A_{OV} of not greater than about 1.0 m³.

According to still another aspect, a portable anesthesia apparatus may include an anesthesia transportation cart, a hospital-grade anesthesia machine mounted on the anesthesia transportation cart, and a hospital-grade CO₂ absorber mounted on the anesthesia transportation cart. The portable anesthesia apparatus may have an A_{H} of not greater than about 1500 mm. The portable anesthesia apparatus may further have an A_{BW} of not greater than about 800 mm. The portable anesthesia apparatus ay further have an A_{BD} of not greater than about 1000 mm.

According to yet another aspect, a portable anesthesia transportation cart may include a portable base, a mounting post extending up from the portable base, a CO₂ absorber mount attached to the mounting post, an equipment shelf attached to the mounting post, and an anesthesia machine mount attached to the equipment shelf. The portable anesthesia transportation cart may have has a C_{FA} of not greater than about 1.0 m².

According to yet another aspect, a portable anesthesia transportation cart may include a portable base, a mounting post extending up from the portable base, a CO₂ absorber mount attached to the mounting post, an equipment shelf attached to the mounting post, and an anesthesia machine mount attached to the equipment shelf. The portable anesthesia transportation cart may have a C_{OV} of not greater than about 1.0 m³.

According to yet another aspect, a portable anesthesia transportation cart may include a portable base, a mounting post extending up from the portable base, a CO₂ absorber mount attached to the mounting post, an equipment shelf attached to the mounting post, and an anesthesia machine mount attached to the equipment shelf. The portable anesthesia transportation cart may have a C_{H} of not greater than about 1500 mm. The portable anesthesia transportation cart may further have a C_{BW} of not greater than about 800 mm. The portable anesthesia transportation cart may further have a C_{BD} of not greater than about 1000 mm.

### BRIEF DESCRIPTION OF THE DRAWINGS

Embodiments are illustrated by way of example and are not limited to the accompanying figures.

FIG. 1 includes an illustration of a perspective view of an embodiment of a portable anesthesia apparatus;

FIG. 2 includes an illustration of a perspective view of an embodiment of an anesthesia transportation cart;

FIG. 3 includes an illustration of a perspective view of another embodiment of a portable anesthesia apparatus;

FIG. 4 includes an illustration of a perspective view of another embodiment of an anesthesia transportation cart;

FIG. 5 includes an illustration of a perspective view of another embodiment of a portable anesthesia apparatus;

FIG. 6 includes an illustration of a perspective view of another embodiment of a portable anesthesia apparatus;

FIG. 7 includes an illustration of a perspective view of another embodiment of a portable anesthesia apparatus;

FIG. 8 includes an illustration of a perspective view of another embodiment of a portable anesthesia apparatus;

FIG. 9 includes an illustration of a perspective view of another embodiment of an anesthesia transportation cart; and

FIG. 10 includes an illustration of a perspective view of another embodiment of an anesthesia transportation cart.

Skilled artisans appreciate that elements in the figures are illustrated for simplicity and clarity and have not necessarily been drawn to scale.

### DETAILED DESCRIPTION

The following discussion will focus on specific implementations and embodiments of the teachings. The detailed description is provided to assist in describing certain embodiments and should not be interpreted as a limitation on the scope or applicability of the disclosure or teachings. It will be appreciated that other embodiments can be used based on the disclosure and teachings as provided herein.

The terms “comprises,” “comprising,” “includes,” “including,” “has,” “having” or any other variation thereof, are intended to cover a non-exclusive inclusion. For example, a method, article, or apparatus that comprises a list of features is not necessarily limited only to those features but may include other features not expressly listed or inherent to such method, article, or apparatus. Further, unless expressly stated to the contrary, “or” refers to an inclusive-or and not to an exclusive-or. For example, a condition A or B is satisfied by any one of the following: A is true (or present) and B is false (or not present), A is false (or not present) and B is true (or present), and both A and B are true (or present).

Also, the use of “a” or “an” is employed to describe elements and components described herein. This is done merely for convenience and to give a general sense of the scope of the invention. This description should be read to include one, at least one, or the singular as also including the plural, or vice versa, unless it is clear that it is meant otherwise. For example, when a single item is described herein, more than one item may be used in place of a single item. Similarly, where more than one item is described herein, a single item may be substituted for that more than one item.

Embodiments described herein are generally directed to a portable anesthesia apparatus having full anesthetic function and being configured to have a minimal footprint area, a minimal occupying volume or both. According to certain embodiments, the portable anesthesia apparatus may includes a transportation cart, a hospital-grade anesthesia machine mounted on the transportation cart, and a hospital-grade CO₂ absorber mounted on the transportation cart. For purposes of embodiments described herein, full anesthetic function may be defined as the ability to facilitate the application of general anesthesia to a patient through the use of a hospital-grade anesthesia machine and a hospital-grade carbon dioxide (CO₂) absorber.

These concepts are better understood in view of the embodiments described below that illustrate and do not limit the scope of the present disclosure.

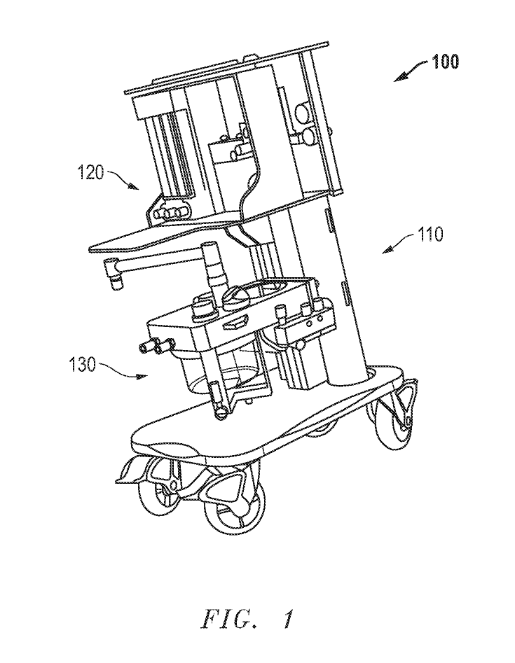

FIG. 1 includes an illustration of a perspective view of an embodiment of a portable anesthesia apparatus 100. According to particular embodiments described herein, and as shown in FIG. 1, a portable anesthesia apparatus 100 may include an anesthesia transportation cart 110, a hospital-grade anesthesia machine 120 mounted on the anesthesia transportation cart 110 and a hospital-grade CO₂ absorber 130 mounted on the anesthesia transportation cart 110.

For purposes of embodiments described herein, an anesthesia machine may be considered a hospital-grade anesthesia machine 120 if it is capable of administering hospital-grade anesthesia. According to still other embodiments, the hospital-grade anesthesia machine 120 may be an FDA approved anesthesia machine. According to certain embodiments, the hospital-grade anesthesia machine 120 may be a three gas machine. According to still other embodiments, the hospital-grade anesthesia machine 120 may include a mechanical anti-hypoxic device. According to yet other embodiments, the hospital-grade anesthesia machine 120 may include an air/N₂O interlock. According to still other embodiments, the hospital-grade anesthesia machine 120 may include ventilator options. According to yet other embodiments, the hospital-grade anesthesia machine 120 may include a vaporizer mounting system. Accordingly to yet other embodiments, the hospital-grade anesthesia machine 120 may include flush valves. According to still other embodiments, the hospital-grade anesthesia machine 120 may accommodate low flow operation.

For purposes of embodiments described herein, a CO₂ absorber may be considered a hospital-grade CO₂ absorber 130 if it is capable of facilitating the administration of hospital grade anesthesia. According to still other embodiments, a hospital-grade CO₂ absorber 130 may be an FDA approved CO₂ absorber. According to yet other embodiments, a hospital-grade CO₂ absorber may include a high capacity CO₂ absorber/ventilator interface. According to still other embodiments, a hospital-grade CO₂ absorber may include be autoclavable (i.e., it may be sterilized). According to yet other embodiments, a hospital-grade CO₂ absorber may include Integrated spirometry and oxygen sensors. According to yet other embodiments, a hospital-grade CO₂ absorber may include changeable absorber material.

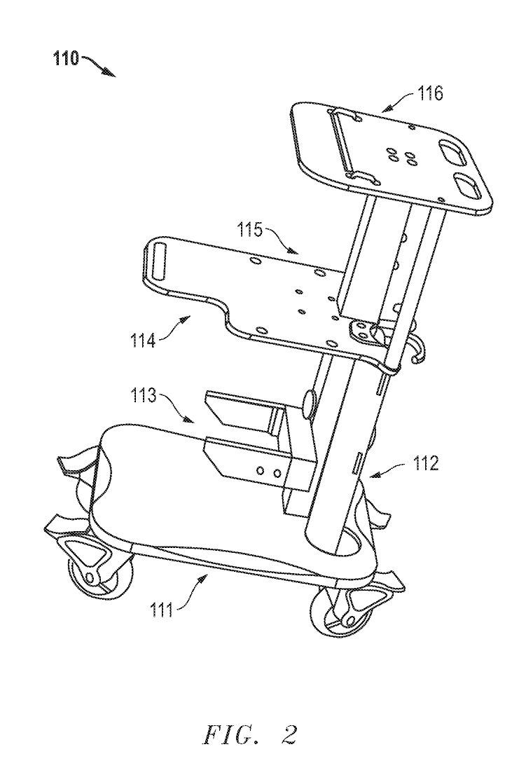

FIG. 2 includes an illustration of a perspective view of the anesthesia transportation cart 110. According to particular embodiments described herein, and as shown in FIG. 2, the anesthesia transportation cart 110 may include a portable base 111, a mounting post 112 extending up from the portable base 111, a CO₂ absorber mount 113 attached to the mounting post 112, an lower equipment shelf 114 attached to the mounting post 112, an anesthesia machine mount 115 attached to the equipment shelf 114, and an upper equipment shelf 116 attached to the mounting post 112.

According to particular embodiments described herein, the portable anesthesia apparatus 100, as shown in FIG. 1, may have a particular maximum apparatus footprint area A_{FA}. For purposes of embodiments described herein, the A_{FA} of a portable anesthesia apparatus 100 may be defined as an area of the smallest rectangle that can fully encompasses the downward projection of the anesthesia apparatus 100 onto the ground, where the anesthesia apparatus is in a generally upright position relative to the ground. According to particular embodiments, the portable anesthesia apparatus 100 may have a maximum A_{FA} of not greater than about 1.0 m², such as, not greater than about 0.95 m² or not greater than about 0.85 m² or not greater than about 0.80 m² or not greater than about 0.75 m² or not greater than about 0.7 m² or not greater than about 0.65 m² or not greater than about 0.6 m² or not greater than about 0.55 m² or not greater than about 0.5 m². According to yet other embodiments, the portable anesthesia apparatus 100 may have a maximum A_{FA} of at least about 0.1 m², such as, at least about 0.15 m² or at least about 0.2 m² or at least about 0.25 m² or at least about or even at least about 0.3 m². It will be appreciated that the maximum A_{FA} of the portable anesthesia apparatus 100 may be within a range between any of the values noted above. It will be further appreciated that the maximum A_{FA} of the portable anesthesia apparatus 100 may be any value between any of the values noted above.

According to still other embodiments described herein, the anesthesia transportation cart 110, as shown in FIGS. 1 and 2, may have a particular maximum cart footprint area C_{FA}. For purposes of embodiments described herein, the C_{FA} of an anesthesia transportation cart 110 may be defined as an area of the smallest rectangle that can fully encompasses the downward projection of the anesthesia transportation cart 110 onto the ground, where the anesthesia transportation cart 110 is in a generally upright position relative to the ground. According to particular embodiments, the anesthesia transportation cart 110 may have a maximum C_{FA} of not greater than about 1.0 m², such as, not greater than about 0.95 m² or not greater than about 0.85 m² or not greater than about 0.80 m² or not greater than about 0.75 m² or not greater than about 0.7 m² or not greater than about 0.65 m² or not greater than about 0.6 m² or not greater than about 0.55 m² or not greater than about 0.5 m². According to yet other embodiments, the anesthesia transportation cart 110 may have a maximum C_{FA} of at least about 0.1 m², such as, at least about 0.15 m² or at least about 0.2 m² or at least about 0.25 m² or at least about or even at least about 0.3 m². It will be appreciated that the maximum C_{FA} of the anesthesia transportation cart 110 may be within a range between any of the values noted above. It will be further appreciated that the maximum C_{FA} of the anesthesia transportation cart 110 may be any value between any of the values noted above.

According to particular embodiments described herein, the portable anesthesia apparatus 100, as shown in FIG. 1, may have a particular apparatus occupying volume A_{OV}. For purposes of embodiments described herein, the A_{OV} of a portable anesthesia apparatus 100 may be defined as the height of the anesthesia transportation cart 110 multiplied by the A_{FA} of the portable anesthesia apparatus 100. According to particular embodiments, the portable anesthesia apparatus 100 may have an A_{OV} of not greater than about 1.0 m³, such as, not greater than about 0.95 m³ or not greater than about 0.85 m³ or not greater than about 0.80 m³ or not greater than about 0.75 m³ or not greater than about 0.7 m³ or not greater than about 0.65 m³ or not greater than about 0.6 m³ or not greater than about 0.55 m³ or not greater than about 0.5 m³. According to still other embodiments, the portable anesthesia apparatus 100 may have an A_{OV} of at least about at least about 0.1 m³, such as, at least about 0.15 m³ or at least about 0.2 m³ or at least about 0.25 m³ or at least about or even at least about 0.3 m³. It will be appreciated that the A_{OV} of the portable anesthesia apparatus 100 may be within a range between any of the values noted above. It will be further appreciated that the A_{OV} of the portable anesthesia apparatus 100 may be any value between any of the values noted above.

According to still other embodiments described herein, the anesthesia transportation cart 110, as shown in FIGS. 1 and 2, may have a particular cart occupying volume C_{OV}. For purposes of embodiments described herein, the C_{OV} of an anesthesia transportation cart 110 may be defined as the height of the anesthesia transportation cart 110 multiplied by the C_{FA} of the transportation cart 110. According to particular embodiments, the anesthesia transportation cart 110 may have a C_{OV} of not greater than about 1.0 m³, such as, not greater than about 0.95 m³ or not greater than about 0.85 m³ or not greater than about 0.80 m³ or not greater than about 0.75 m³ or not greater than about 0.7 m³ or not greater than about 0.65 m³ or not greater than about 0.6 m³ or not greater than about 0.55 m³ or not greater than about 0.5 m³. According to still other embodiments, the anesthesia transportation cart 110 may have a C_{OV} of at least about at least about 0.1 m³, such as, at least about 0.15 m³ or at least about 0.2 m³ or at least about 0.25 m³ or at least about or even at least about 0.3 m³. It will be appreciated that the C_{OV} of the anesthesia transportation cart 110 may be within a range between any of the values noted above. It will be further appreciated that the C_{OV} of the transportation cart 110 may be any value between any of the values noted above.

According to particular embodiments described herein, the portable anesthesia apparatus 100, as shown in FIG. 1, may have a particular apparatus height A_{H}. For purposes of embodiments described herein, the A_{H} of the portable anesthesia apparatus 100 may be defined as the height of the anesthesia transportation cart 110 of the portable anesthesia apparatus 100 as measured from the ground when the anesthesia apparatus 100 is in a generally upright position relative to the ground. According to certain embodiments, the portable anesthesia apparatus 100 may have an A_{H} of not greater than about 1500 mm, such as, not greater than about 1475 mm or not greater than about 1450 mm or not greater than about 1425 mm or not greater than about 1400 mm or not greater than about 1375 mm or not greater than about 1350 mm or not greater than about 1325 mm or not greater than about 1300 mm or not greater than about 1275 mm or not greater than about 1250 mm or not greater than about 1225 mm or not greater than about 1200 mm or not greater than about 1175 mm or not greater than about 1150 mm or not greater than about 1125 mm or not greater than about 1100 mm or not greater than about 1075 mm or not greater than about 1050 mm or not greater than about 1025 mm or even not greater than about 1000 mm. According to still other embodiments, the portable anesthesia apparatus 100 may have an A_{H} of at least about 500 mm, such as, at least about 525 mm or at least about 550 mm or at least about 575 mm or at least about 600 mm or at least about 625 mm or at least about 650 mm or at least about 675 mm or at least about 700 mm or at least about 725 mm or at least about 750 mm or at least about 775 mm or at least about 800 mm or at least about 825 mm or at least about 850 mm or at least about 875 mm or even at least about 900 mm. It will be appreciated that the A_{H} of the portable anesthesia apparatus 100 may be within a range between any of the values noted above. It will be further appreciated that the A_{H} of the portable anesthesia apparatus 100 may be any value between any of the values noted above.

According to still other embodiments described herein, the anesthesia transportation cart 110, as shown in FIGS. 1 and 2, may have a particular cart height C_{H}. For purposes of embodiments described herein, the C_{H} of the anesthesia transportation cart 110 may be defined as the height of the anesthesia transportation cart 110 as measured from the ground when the anesthesia transportation cart 110 is in a generally upright position relative to the ground. According to certain embodiments, the anesthesia transportation cart 110 may have an C_{H} of not greater than about 1500 mm, such as, not greater than about 1475 mm or not greater than about 1450 mm or not greater than about 1425 mm or not greater than about 1400 mm or not greater than about 1375 mm or not greater than about 1350 mm or not greater than about 1325 mm or not greater than about 1300 mm or not greater than about 1275 mm or not greater than about 1250 mm or not greater than about 1225 mm or not greater than about 1200 mm or not greater than about 1175 mm or not greater than about 1150 mm or not greater than about 1125 mm or not greater than about 1100 mm or not greater than about 1075 mm or not greater than about 1050 mm or not greater than about 1025 mm or even not greater than about 1000 mm. According to still other embodiments, the anesthesia transportation cart 110 may have an C_{H} of at least about 500 mm, such as, at least about 525 mm or at least about 550 mm or at least about 575 mm or at least about 600 mm or at least about 625 mm or at least about 650 mm or at least about 675 mm or at least about 700 mm or at least about 725 mm or at least about 750 mm or at least about 775 mm or at least about 800 mm or at least about 825 mm or at least about 850 mm or at least about 875 mm or even at least about 900 mm. It will be appreciated that the C_{H} of the anesthesia transportation cart 110 may be within a range between any of the values noted above. It will be further appreciated that the C_{H} of the anesthesia transportation cart 110 may be any value between any of the values noted above.

According to particular embodiments described herein, the portable anesthesia apparatus 100, as shown in FIG. 1, may have a particular apparatus base width A_{BW}. For purposes of embodiments described herein, the A_{BW} of the portable anesthesia apparatus 100 may be defined as the width of the portable base 111 of the anesthesia transportation cart 110. According to certain embodiments, the portable anesthesia apparatus 100 may have an A_{BW} of not greater than about 800 mm, such as, not greater tan about 775 mm or not greater than about 750 mm or not greater than about 725 mm or not greater than about 700 mm or not greater than about 675 mm or not greater than about 650 mm or not greater than about 625 mm or not greater than about 600 mm or not greater than about 575 mm or not greater than about 550 mm or not greater than about 525 mm or even not greater than about 500. According to yet other embodiments, the portable anesthesia apparatus 100 may have a A_{BW} of at least about 200 mm, such as, at least about 225 mm or at least about 250 mm or at least about 275 mm or at least about 300 mm or at least about 325 mm or at least about 350 mm or at least about 375 mm or at least about 400 mm or at least about 425 mm or even at least about 450 mm. It will be appreciated that the A_{BW} of the portable anesthesia apparatus 100 may be within a range between any of the values noted above. It will be further appreciated that the A_{BW} of the portable anesthesia apparatus 100 may be any value between any of the values noted above.

According to still other embodiments described herein, the anesthesia transportation cart 110, as shown in FIGS. 1 and 2, may have a particular cart base width C_{BW}. For purposes of embodiments described herein, the C_{BW} of the anesthesia transportation cart 110 may be defined as the width of the portable base 111 of the anesthesia transportation cart 110. According to certain embodiments, the anesthesia transportation cart 110 may have an C_{BW} of not greater than about 800 mm, such as, not greater tan about 775 mm or not greater than about 750 mm or not greater than about 725 mm or not greater than about 700 mm or not greater than about 675 mm or not greater than about 650 mm or not greater than about 625 mm or not greater than about 600 mm or not greater than about 575 mm or not greater than about 550 mm or not greater than about 525 mm or even not greater than about 500. According to yet other embodiments, the anesthesia transportation cart 110 may have a C_{BW} of at least about 200 mm, such as, at least about 225 mm or at least about 250 mm or at least about 275 mm or at least about 300 mm or at least about 325 mm or at least about 350 mm or at least about 375 mm or at least about 400 mm or at least about 425 mm or even at least about 450 mm. It will be appreciated that the C_{BW} of the anesthesia transportation cart 110 may be within a range between any of the values noted above. It will be further appreciated that the C_{BW} of the anesthesia transportation cart 110 may be any value between any of the values noted above.

According to particular embodiments described herein, the portable anesthesia apparatus 100, as shown in FIG. 1, may have a particular apparatus base depth A_{BD}. For purposes of embodiments described herein, the A_{BD} of the portable anesthesia apparatus 100 may be defined as the depth of the portable base 111 of the anesthesia transportation cart 110. According to certain embodiments, the portable anesthesia apparatus 100 may have an A_{BD} of not greater than about 1000 mm, such as, not greater than about 975 mm or not greater than about 950 mm or not greater than about 925 mm or not greater than about 900 mm or not greater than about 975 mm or not greater than about 950 mm or not greater than about 925 mm or not greater than about 900 mm or not greater than about 875 mm or not greater than about 850 mm or not greater than about 825 mm or even not greater than about 800 mm or even not greater than about 775 mm or even not greater than about 750 mm or even not greater than about 725 mm or even not greater than about 700. According to yet other embodiments, the portable anesthesia apparatus 100 may have an A_{BD} of at least about 300 mm, such as, at least about 325 mm or at least about 350 mm or at least about 375 mm or at least about 400 mm or at least about 425 mm or at least about 450 mm or at least about 475 mm or at least about 500 mm or at least about 525 mm or even at least about 550 mm. It will be appreciated that the A_{BD} of the portable anesthesia apparatus 100 may be within a range between any of the values noted above. It will be further appreciated that the A_{BD} of the portable anesthesia apparatus 100 may be any value between any of the values noted above.

According to still other embodiments described herein, the anesthesia transportation cart 110, as shown in FIGS. 1 and 2, may have a particular cart base depth C_{BD}. For purposes of embodiments described herein, the C_{BD} of the anesthesia transportation cart 110 may be defined as the depth of the portable base 111 of the anesthesia transportation cart 110. According to certain embodiments, the anesthesia transportation cart 110 may have a C_{BD} of not greater than about 1000 mm, such as, not greater than about 975 mm or not greater than about 950 mm or not greater than about 925 mm or not greater than about 900 mm or not greater than about 975 mm or not greater than about 950 mm or not greater than about 925 mm or not greater than about 900 mm or not greater than about 875 mm or not greater than about 850 mm or not greater than about 825 mm or even not greater than about 800 mm or even not greater than about 775 mm or even not greater than about 750 mm or even not greater than about 725 mm or even not greater than about 700. According to yet other embodiments, the anesthesia transportation cart 110 may have an C_{BD} of at least about 300 mm, such as, at least about 325 mm or at least about 350 mm or at least about 375 mm or at least about 400 mm or at least about 425 mm or at least about 450 mm or at least about 475 mm or at least about 500 mm or at least about 525 mm or even at least about 550 mm. It will be appreciated that the C_{BD} of the anesthesia transportation cart 110 may be within a range between any of the values noted above. It will be further appreciated that the C_{BD} of the anesthesia transportation cart 110 may be any value between any of the values noted above.

According to particular embodiments described herein, the portable anesthesia apparatus 100, as shown in FIG. 1, may have a particular apparatus weight A_{W}. According to certain embodiments, the portable anesthesia apparatus 100 may have an A_{W} of not greater than about 210 kg, such as, not greater than about 205 kg or not greater than about 200 kg or not greater than about 195 kg or not greater than about 190 kg or not greater than about 185 kg or not greater than about 180 kg or not greater than about 175 kg or not greater than about 170 kg or not greater than about 165 kg or not greater than about 160 kg or not greater than about 155 kg or even not greater than about 150 kg. According to yet other embodiments, the portable anesthesia apparatus 100 may have a A_{W} of at least about 50 kg, such as, at least about 55 kg or at least about 60 kg or at least about 65 kg or at least about 70 kg or at least about 75 kg or at least about 80 kg or at least about 85 kg or at least about 90 kg or at least about 95 kg or even at least about 100 kg. It will be appreciated that the A_{W} of the portable anesthesia apparatus 100 may be within a range between any of the values noted above. It will be further appreciated that the A_{W} of the portable anesthesia apparatus 100 may be any value between any of the values noted above.

According to still other embodiments described herein, the anesthesia transportation cart 110, as shown in FIGS. 1 and 2, may have a particular cart weight C_{W}. According to certain embodiments, the anesthesia transportation cart 110 may have an C_{W} of not greater than about 210 kg, such as, not greater than about 205 kg or not greater than about 200 kg or not greater than about 195 kg or not greater than about 190 kg or not greater than about 185 kg or not greater than about 180 kg or not greater than about 175 kg or not greater than about 170 kg or not greater than about 165 kg or not greater than about 160 kg or not greater than about 155 kg or even not greater than about 150 kg. According to yet other embodiments, the anesthesia transportation cart 110 may have a C_{W} of at least about 50 kg, such as, at least about 55 kg or at least about 60 kg or at least about 65 kg or at least about 70 kg or at least about 75 kg or at least about 80 kg or at least about 85 kg or at least about 90 kg or at least about 95 kg or even at least about 100 kg. It will be appreciated that the C_{W} of the anesthesia transportation cart 110 may be within a range between any of the values noted above. It will be further appreciated that the C_{W} of the anesthesia transportation cart 110 may be any value between any of the values noted above.

According to still other embodiments described herein, the portable base 111 of the anesthesia transportation cart 110, as shown in FIG. 2, may have a particular thickness B_{T}. For example, the portable base 111 may have a B_{T} of not greater than about 200 mm, such as, not greater than about 195 mm or not greater than about 190 mm or not greater than about 185 mm or not greater than about 180 mm or not greater than about 175 mm or not greater than about 170 mm or not greater than about 165 mm or not greater than about 160 mm or not greater than about 155 mm or not greater than about 150 mm or not greater than about 145 mm or not greater than about 140 mm or not greater than about 135 mm or not greater than about 130 mm or not greater than about 125 mm or not greater than about 120 mm or not greater than about 115 mm or not greater than about 110 mm or not greater than about 105 mm or even not greater than about 100 mm. According to still other embodiments, the portable base 111 may have a B_{T} of at least about 20 mm, such as, at least about 25 mm or at least about 30 mm or at least about 35 mm or at least about 40 mm or at least about 45 mm or at least about 50 mm or at least about 55 mm or at least about 60 mm or at least about 65 mm or at least about 70 mm or at least about 75 mm or at least about 80 mm or at least about 85 mm or at least about 90 mm or even at least about 95 mm. It will be appreciated that the B_{T} of the portable base 111 may be within a range between any of the values noted above. It will be further appreciated that the B_{T} of the portable base 111 may be any value between any of the values noted above.

According to still other embodiments described herein, the portable base 111 of the anesthesia transportation cart 110, as shown in FIG. 2, may have a particular base width B_{W}. According to certain embodiments, the portable base 111 may have a B_{W} of not greater than about 800 mm, such as, not greater tan about 775 mm or not greater than about 750 mm or not greater than about 725 mm or not greater than about 700 mm or not greater than about 675 mm or not greater than about 650 mm or not greater than about 625 mm or not greater than about 600 mm or not greater than about 575 mm or not greater than about 550 mm or not greater than about 525 mm or even not greater than about 500. According to yet other embodiments, the portable base 111 may have a B_{W} of at least about 200 mm, such as, at least about 225 mm or at least about 250 mm or at least about 275 mm or at least about 300 mm or at least about 325 mm or at least about 350 mm or at least about 375 mm or at least about 400 mm or at least about 425 mm or even at least about 450 mm. It will be appreciated that the B_{W} of the portable base 111 may be within a range between any of the values noted above. It will be further appreciated that the B_{W} of the portable base 111 may be any value between any of the values noted above.

According to still other embodiments described herein, the portable base 111 of the anesthesia transportation cart 110, as shown in FIG. 2, may have a particular base depth B_{D}. According to certain embodiments, the portable base 111 may have a B_{D} of not greater than about 1000 mm, such as, not greater than about 975 mm or not greater than about 950 mm or not greater than about 925 mm or not greater than about 900 mm or not greater than about 975 mm or not greater than about 950 mm or not greater than about 925 mm or not greater than about 900 mm or not greater than about 875 mm or not greater than about 850 mm or not greater than about 825 mm or even not greater than about 800 mm or even not greater than about 775 mm or even not greater than about 750 mm or even not greater than about 725 mm or even not greater than about 700. According to yet other embodiments, the portable base 111 may have an B_{D} of at least about 300 mm, such as, at least about 325 mm or at least about 350 mm or at least about 375 mm or at least about 400 mm or at least about 425 mm or at least about 450 mm or at least about 475 mm or at least about 500 mm or at least about 525 mm or even at least about 550 mm. It will be appreciated that the B_{D} of the portable base 111 may be within a range between any of the values noted above. It will be further appreciated that the B_{D} of the portable base 111 may be any value between any of the values noted above.

According to still other embodiments described herein, the mounting post 112 of the anesthesia transportation cart 110, as shown in FIG. 2, may have a particular height MP_{H}. For example, the mounting post 112 may have a MP_{H} of not greater than about 1500 mm, such as, not greater than about 1475 mm or not greater than about 1450 mm or not greater than about 1425 mm or not greater than about 1400 mm or not greater than about 1375 mm or not greater than about 1350 mm or not greater than about 1325 mm or not greater than about 1300 mm or not greater than about 1275 mm or not greater than about 1250 mm or not greater than about 1225 mm or not greater than about 1200 mm or not greater than about 1175 mm or not greater than about 1150 mm or not greater than about 1125 mm or not greater than about 1100 mm or not greater than about 1075 mm or not greater than about 1050 mm or not greater than about 1025 mm or even not greater than about 1000 mm. According to still other embodiments, the mounting post 112 may have a MP_{H} of at least about 500 mm, such as, at least about 525 mm or at least about 550 mm or at least about 575 mm or at least about 600 mm or at least about 625 mm or at least about 650 mm or at least about 675 mm or at least about 700 mm or at least about 725 mm or at least about 750 mm or at least about 775 mm or at least about 800 mm or at least about 825 mm or at least about 850 mm or at least about 875 mm or even at least about 900 mm. It will be appreciated that the MP_{H} of the mounting post 112 may be within a range between any of the values noted above. It will be further appreciated that the MP_{H} of the mounting post 112 may be any value between any of the values noted above.

According to still other embodiments described herein, the mounting post 112 of the anesthesia transportation cart 110, as shown in FIG. 2, may have a particular mounting post width MP_{W}. According to certain embodiments, the mounting post 112 may have a MP_{W} of not greater than about 180 mm, such as, not greater tan about 170 mm or not greater than about 160 mm or not greater than about 150 mm or not greater than about 140 mm or not greater than about 130 mm or even not greater than about 120. According to yet other embodiments, the mounting post 112 may have a MP_{W} of at least about 50 mm, such as, at least about 60 mm or at least about 70 mm or at least about 80 mm or at least about 90 mm or at least about 100 mm or even at least about 110 mm. It will be appreciated that the MP_{W} of the mounting post 112 may be within a range between any of the values noted above. It will be further appreciated that the MP_{W} of the mounting post 112 may be any value between any of the values noted above.

According to still other embodiments described herein, the mounting post 112 of the anesthesia transportation cart 110, as shown in FIG. 2, may have a particular mounting post depth MP_{D}. According to certain embodiments, the mounting post 112 may have a MP_{D} of not greater than about 60 mm, such as, not greater than about 55 mm or not greater than about 50 mm or not greater than about 45 mm or not greater than about 40 mm or not greater than about 35 mm or even not greater than about 30 mm. According to yet other embodiments, the mounting post 112 may have an MP_{D} of at least about 10 mm, such as, at least about 15 mm or at least about 20 mm or even at least about 25 mm. It will be appreciated that the MP_{D} of the mounting post 112 may be within a range between any of the values noted above. It will be further appreciated that the MP_{D} of the mounting post 112 may be any value between any of the values noted above.

According to still other embodiments described herein, the CO₂ absorber mount 113, as shown in FIG. 2, may be configured to mount a hospital-grade CO₂ absorber.

According to still other embodiments described herein, the lower equipment shelf 114 of the anesthesia transportation cart 110, as shown in FIG. 2, may have a particular thickness LES_{T}. For example, the lower equipment shelf 114 may have a LES_{T} of not greater than about 200 mm, such as, not greater than about 195 mm or not greater than about 190 mm or not greater than about 185 mm or not greater than about 180 mm or not greater than about 175 mm or not greater than about 170 mm or not greater than about 165 mm or not greater than about 160 mm or not greater than about 155 mm or not greater than about 150 mm or not greater than about 145 mm or not greater than about 140 mm or not greater than about 135 mm or not greater than about 130 mm or not greater than about 125 mm or not greater than about 120 mm or not greater than about 115 mm or not greater than about 110 mm or not greater than about 105 mm or even not greater than about 100 mm. According to still other embodiments, the lower equipment shelf 114 may have a LES_{T} of at least about 20 mm, such as, at least about 25 mm or at least about 30 mm or at least about 35 mm or at least about 40 mm or at least about 45 mm or at least about 50 mm or at least about 55 mm or at least about 60 mm or at least about 65 mm or at least about 70 mm or at least about 75 mm or at least about 80 mm or at least about 85 mm or at least about 90 mm or even at least about 95 mm. It will be appreciated that the LES_{T} of the lower equipment shelf 114 may be within a range between any of the values noted above. It will be further appreciated that the LES_{T} of the lower equipment shelf 114 may be any value between any of the values noted above.

According to still other embodiments described herein, the lower equipment shelf 114 of the anesthesia transportation cart 110, as shown in FIG. 2, may have a particular shelf width LES_{W}. According to certain embodiments, the lower equipment shelf 114 may have a LES_{W} of not greater than about 800 mm, such as, not greater tan about 775 mm or not greater than about 750 mm or not greater than about 725 mm or not greater than about 700 mm or not greater than about 675 mm or not greater than about 650 mm or not greater than about 625 mm or not greater than about 600 mm or not greater than about 575 mm or not greater than about 550 mm or not greater than about 525 mm or even not greater than about 500. According to yet other embodiments, the lower equipment shelf 114 may have a LES_{W} of at least about 200 mm, such as, at least about 225 mm or at least about 250 mm or at least about 275 mm or at least about 300 mm or at least about 325 mm or at least about 350 mm or at least about 375 mm or at least about 400 mm or at least about 425 mm or even at least about 450 mm. It will be appreciated that the LES_{W} of the lower equipment shelf 114 may be within a range between any of the values noted above. It will be further appreciated that the LES_{W} of the lower equipment shelf 114 may be any value between any of the values noted above.

According to still other embodiments described herein, the lower equipment shelf 114 of the anesthesia transportation cart 110, as shown in FIG. 2, may have a particular shelf depth LES_{D}. According to certain embodiments, the lower equipment shelf 114 may have a LES_{D} of not greater than about 1000 mm, such as, not greater than about 975 mm or not greater than about 950 mm or not greater than about 925 mm or not greater than about 900 mm or not greater than about 975 mm or not greater than about 950 mm or not greater than about 925 mm or not greater than about 900 mm or not greater than about 875 mm or not greater than about 850 mm or not greater than about 825 mm or even not greater than about 800 mm or even not greater than about 775 mm or even not greater than about 750 mm or even not greater than about 725 mm or even not greater than about 700. According to yet other embodiments, the lower equipment shelf 114 may have an LES_{D} of at least about 300 mm, such as, at least about 325 mm or at least about 350 mm or at least about 375 mm or at least about 400 mm or at least about 425 mm or at least about 450 mm or at least about 475 mm or at least about 500 mm or at least about 525 mm or even at least about 550 mm. It will be appreciated that the LES_{D} of the lower equipment shelf 114 may be within a range between any of the values noted above. It will be further appreciated that the LES_{D} of the lower equipment shelf 114 may be any value between any of the values noted above.

According to still other embodiments described herein, the anesthesia machine mount 15, as shown in FIG. 1, may be configured to mount a commercial anesthesia machine.

According to still other embodiments described herein, the upper equipment shelf 116 of the anesthesia transportation cart 110, as shown in FIG. 2, may have a particular thickness UES_{T}. For example, the upper equipment shelf 116 may have a UES_{T} of not greater than about 200 mm, such as, not greater than about 195 mm or not greater than about 190 mm or not greater than about 185 mm or not greater than about 180 mm or not greater than about 175 mm or not greater than about 170 mm or not greater than about 165 mm or not greater than about 160 mm or not greater than about 155 mm or not greater than about 150 mm or not greater than about 145 mm or not greater than about 140 mm or not greater than about 135 mm or not greater than about 130 mm or not greater than about 125 mm or not greater than about 120 mm or not greater than about 115 mm or not greater than about 110 mm or not greater than about 105 mm or even not greater than about 100 mm. According to still other embodiments, the upper equipment shelf 116 may have a UES_{T} of at least about 20 mm, such as, at least about 25 mm or at least about 30 mm or at least about 35 mm or at least about 40 mm or at least about 45 mm or at least about 50 mm or at least about 55 mm or at least about 60 mm or at least about 65 mm or at least about 70 mm or at least about 75 mm or at least about 80 mm or at least about 85 mm or at least about 90 mm or even at least about 95 mm. It will be appreciated that the UES_{T} of the upper equipment shelf 116 may be within a range between any of the values noted above. It will be further appreciated that the UES_{T} of the upper equipment shelf 116 may be any value between any of the values noted above.

According to still other embodiments described herein, the upper equipment shelf 116 of the anesthesia transportation cart 110, as shown in FIG. 2, may have a particular shelf width UES_{W}. According to certain embodiments, the upper equipment shelf 116 may have a UES of not greater than about 800 mm, such as, not greater tan about 775 mm or not greater than about 750 mm or not greater than about 725 mm or not greater than about 700 mm or not greater than about 675 mm or not greater than about 650 mm or not greater than about 625 mm or not greater than about 600 mm or not greater than about 575 mm or not greater than about 550 mm or not greater than about 525 mm or even not greater than about 500. According to yet other embodiments, the upper equipment shelf 116 may have a UES _{W} of at least about 200 mm, such as, at least about 225 mm or at least about 250 mm or at least about 275 mm or at least about 300 mm or at least about 325 mm or at least about 350 mm or at least about 375 mm or at least about 400 mm or at least about 425 mm or even at least about 450 mm. It will be appreciated that the UES of the upper equipment shelf 116 may be within a range between any of the values noted above. It will be further appreciated that the UES of the upper equipment shelf 116 may be any value between any of the values noted above.

According to still other embodiments described herein, the upper equipment shelf 116 of the anesthesia transportation cart 110, as shown in FIG. 2, may have a particular shelf depth UES_{D}. According to certain embodiments, the upper equipment shelf 116 may have a UES_{D} of not greater than about 1000 mm, such as, not greater than about 975 mm or not greater than about 950 mm or not greater than about 925 mm or not greater than about 900 mm or not greater than about 975 mm or not greater than about 950 mm or not greater than about 925 mm or not greater than about 900 mm or not greater than about 875 mm or not greater than about 850 mm or not greater than about 825 mm or even not greater than about 800 mm or even not greater than about 775 mm or even not greater than about 750 mm or even not greater than about 725 mm or even not greater than about 700. According to yet other embodiments, the upper equipment shelf 116 may have an UES_{D} of at least about 300 mm, such as, at least about 325 mm or at least about 350 mm or at least about 375 mm or at least about 400 mm or at least about 425 mm or at least about 450 mm or at least about 475 mm or at least about 500 mm or at least about 525 mm or even at least about 550 mm. It will be appreciated that the UES_{D} of the upper equipment shelf 116 may be within a range between any of the values noted above. It will be further appreciated that the UES_{D} of the upper equipment shelf 116 may be any value between any of the values noted above.

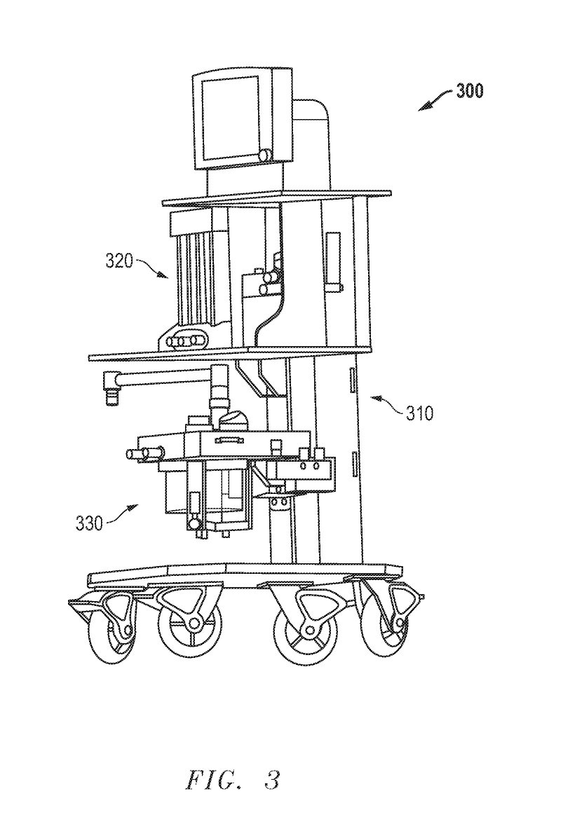

FIG. 3 includes an illustration of a perspective view of another embodiment of a portable anesthesia apparatus 300. According to particular embodiments described herein, and as shown in FIG. 3, a portable anesthesia apparatus 300 may include an anesthesia transportation cart 310, a hospital-grade anesthesia machine 320 mounted on the anesthesia transportation cart 310, a hospital-grade CO₂ absorber 330 mounted on the anesthesia transportation cart 310 and a patient monitor 340 mounted on the anesthesia transportation cart 310.

It will be appreciated that the portable anesthesia apparatus 300 and its components (i.e., the anesthesia transportation cart 310, the hospital-grade anesthesia machine 320, the hospital-grade CO₂ absorber 330) may include or be described as having any of the components and characteristics described in reference the corresponding components shown in FIG. 1 or 2.

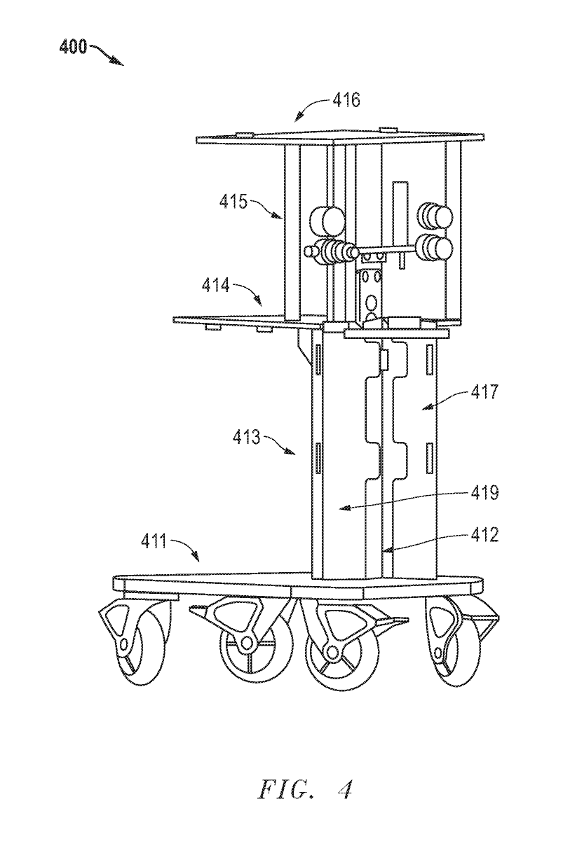

FIG. 4 includes an illustration of a perspective view of another embodiment of an anesthesia transportation cart 410. According to particular embodiments described herein, and as shown in FIG. 4, the anesthesia transportation cart 410 may include a portable base 411, a mounting post 412 extending up from the portable base 411, a CO₂ absorber mount 413 attached to the mounting post 412, an lower equipment shelf 414 attached to the mounting post 412, an anesthesia machine mount 415 attached to the equipment shelf 414, an upper equipment shelf 416 attached to the mounting post 412, an O₂ tank mount 417 attached to the mounting post 412 and a N₂O tank mount 419 attached to the mounting post 412.

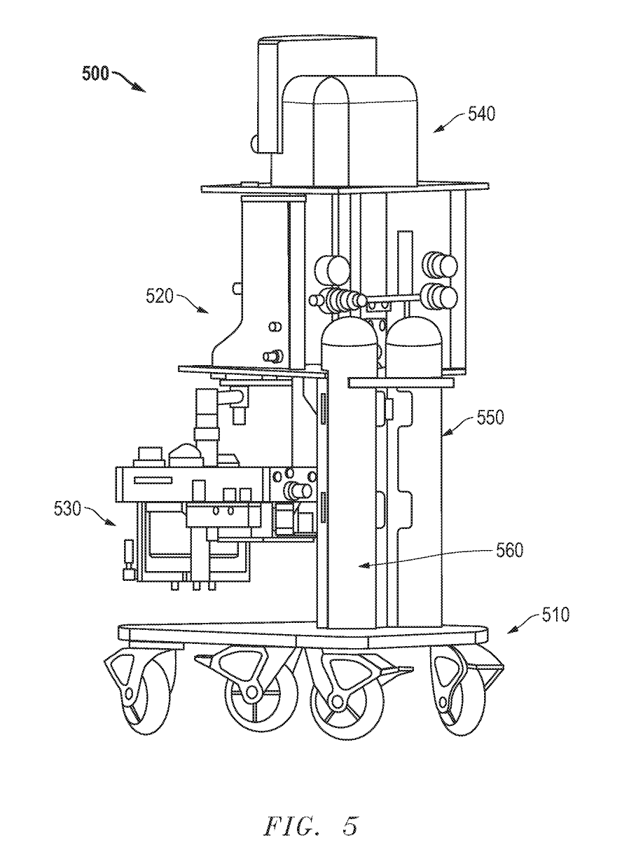

FIG. 5 includes an illustration of a perspective view of another embodiment of a portable anesthesia apparatus 500. According to particular embodiments described herein, and as shown in FIG. 5, a portable anesthesia apparatus 500 may include an anesthesia transportation cart 510, a hospital-grade anesthesia machine 520 mounted on the anesthesia transportation cart 510, a hospital-grade CO₂ absorber 530 mounted on the anesthesia transportation cart 510, a patient monitor 540 mounted on the anesthesia transportation cart 510, an O₂ tank 550 mounted to the anesthesia transportation cart 510 and an N₂O tank 560 mounted to the anesthesia transportation cart 510.

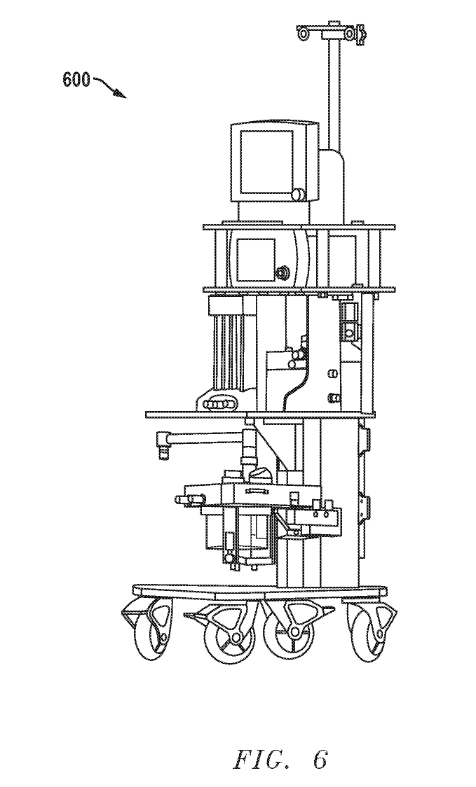

FIG. 6 includes an illustration of a perspective view of another embodiment of a portable anesthesia apparatus 600.

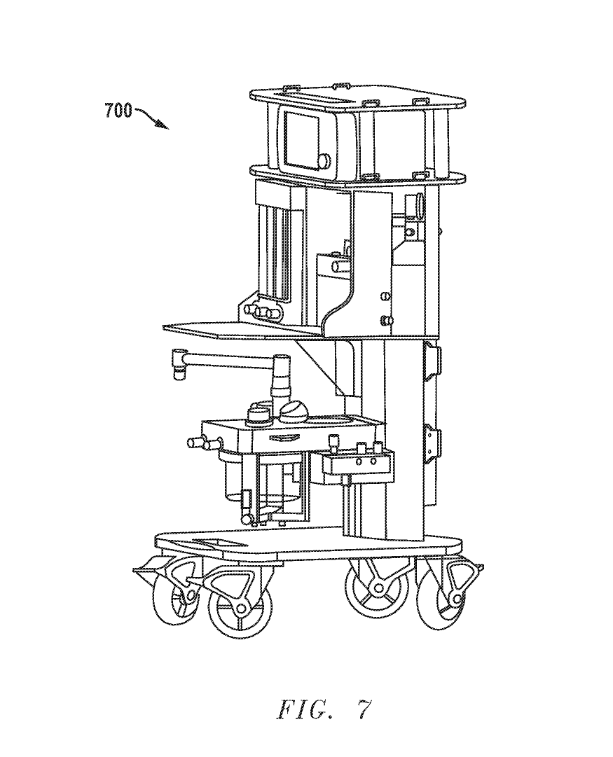

FIG. 7 includes an illustration of a perspective view of another embodiment of a portable anesthesia apparatus 700.

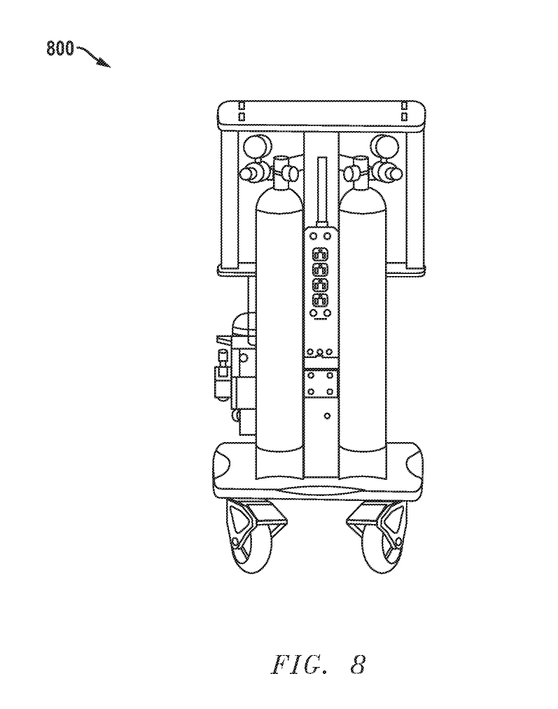

FIG. 8 includes an illustration of a perspective view of another embodiment of a portable anesthesia apparatus 800.

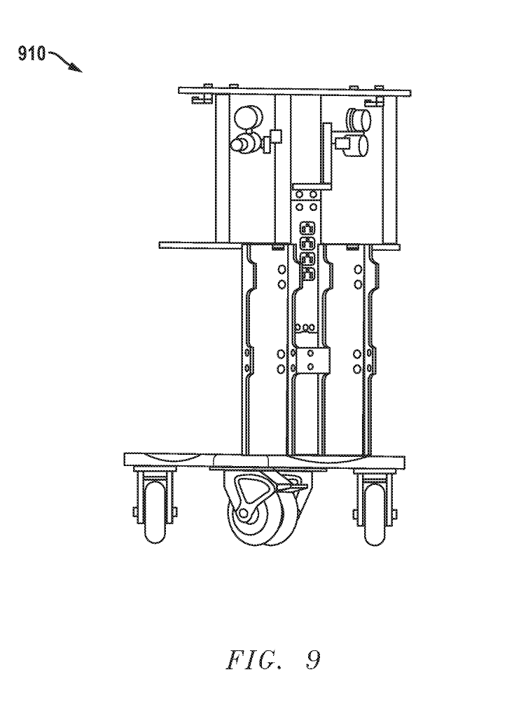

FIG. 9 includes an illustration of a perspective view of another embodiment of an anesthesia transportation cart 910.

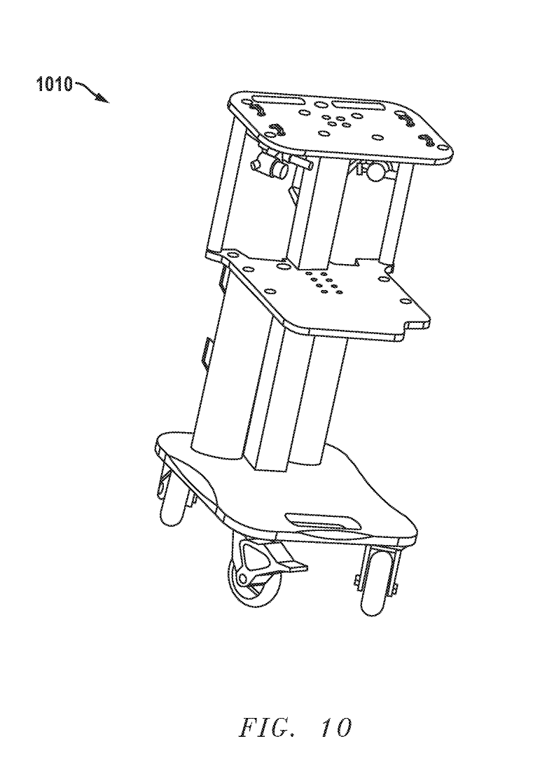

FIG. 10 includes an illustration of a perspective view of another embodiment of an anesthesia transportation cart 1010.

According to still other embodiments, the portable anesthesia apparatuses described herein may be modular, meaning that any components of the apparatus may be disassembled for transportation and reassembled for use. According to still other embodiments, the anesthesia transportation carts described herein may be modular, meaning that any components of the carts may be disassembled for transportation and reassembled for use.

Many different aspects and embodiments are possible. Some of those aspects and embodiments are described below. After reading this specification, skilled artisans will appreciate that those aspects and embodiments are only illustrative and do not limit the scope of the present invention. Embodiments can be in accordance with any one or more of the items as listed below.

Embodiment 1. A portable anesthesia apparatus comprising: an anesthesia transportation cart; a hospital-grade anesthesia machine mounted on the anesthesia transportation cart; and a hospital-grade CO₂ absorber mounted on the anesthesia transportation cart, wherein the portable anesthesia apparatus has an A_{FA} of not greater than about 1.0 m².

Embodiment 2. A portable anesthesia apparatus comprising: an anesthesia transportation cart; a hospital-grade anesthesia machine mounted on the anesthesia transportation car; and a hospital-grade CO₂ absorber mounted on the anesthesia transportation cart, wherein the portable anesthesia apparatus has an A_{OV} of not greater than about 1.0 m³.

Embodiment 3. A portable anesthesia apparatus comprising: an anesthesia transportation cart; a hospital-grade anesthesia machine mounted on the anesthesia transportation car; and a hospital-grade CO₂ absorber mounted on the anesthesia transportation cart, wherein the portable anesthesia apparatus has an A_{H} of not greater than about 1500 mm, wherein the portable anesthesia apparatus has an A_{BW} of not greater than about 800 mm, and wherein the portable anesthesia apparatus has an A_{BD} of not greater than about 1000 mm.

Embodiment 4. The portable anesthesia apparatus of any one of embodiments 1, 2, and 3, wherein the portable anesthesia apparatus has an A_{W} of not greater than about 210 kilograms.

Embodiment 5. The portable anesthesia apparatus of any one of embodiments 1, 2, and 3, wherein the portable anesthesia apparatus has an A_{W} of at least about 50 kilograms.

Embodiment 6. The portable anesthesia apparatus of any one of embodiments 2, and 3, wherein the portable anesthesia apparatus has an A_{FA} of not greater than about 0.5 m².

Embodiment 7. The portable anesthesia apparatus of any one of embodiments 1, 2, and 3, wherein the portable anesthesia apparatus has an A_{FA} of at least about 0.1 m².

Embodiment 8. The portable anesthesia apparatus of any one of embodiments 1, and 3, wherein the portable apparatus has an A_{OV} of not greater than about 0.5 m³.

Embodiment 9. The portable anesthesia apparatus of any one of embodiments 1, 2, and 3, wherein the portable anesthesia apparatus has an A_{OV} of at least about 0.1 m³.

Embodiment 10. The portable anesthesia apparatus of any one of embodiments 1, and 2, wherein the portable apparatus has an A_{H} of not greater than about 1200 mm.

Embodiment 11. The portable anesthesia apparatus of any one of embodiments 1, 2, and 3, wherein the portable anesthesia apparatus has an A_{H} of at least about 500 mm.

Embodiment 12. The portable anesthesia apparatus of any one of embodiments 1, and 2, wherein the portable anesthesia apparatus has an A_{BW} width of not greater than about 550 mm.

Embodiment 13. The portable anesthesia apparatus of any one of embodiments 1, 2, and 3, wherein the portable anesthesia apparatus has an A_{BW} of at least about 200 mm.

Embodiment 14. The portable anesthesia apparatus of any one of embodiments 1, 2, and 3, wherein the portable anesthesia apparatus has an A_{BD} of not greater than about 700 mm.

Embodiment 15. The portable anesthesia apparatus of any one of embodiments 1, 2, and 3, wherein the portable anesthesia apparatus has an A_{BD} of at least about 300 mm.

Embodiment 16. The portable anesthesia apparatus of any one of embodiments 1, 2, and 3, wherein the anesthesia transportation cart comprises: a portable base; a mounting post extending up from the portable base; a CO₂ absorber mount attached to the mounting post; an equipment shelf attached to the mounting post; and an anesthesia machine mount attached to the equipment shelf.

Embodiment 17. The portable anesthesia apparatus of any one of embodiments 1, 2, and 3, wherein the anesthesia transportation cart has a C_{FA} of not greater than about 1.0 m².

Embodiment 18. The portable anesthesia apparatus of any one of embodiments 1, 2, and 3, wherein the anesthesia transportation cart has a C_{FA} of at least about 0.1 m².

Embodiment 19. The portable anesthesia apparatus of any one of embodiments 1, 2, and 3, wherein the anesthesia transportation cart has a C_{OV} of not greater than about 1.0 m³.

Embodiment 20. The portable anesthesia apparatus of any one of embodiments 1, 2, and 3, wherein the anesthesia transportation cart has a C_{OV} of at least about 0.1 m³.

Embodiment 21. The portable anesthesia apparatus of any one of embodiments 1, 2, and 3, wherein the anesthesia transportation cart has a C_{H} of not greater than about 1500 mm.

Embodiment 22. The portable anesthesia apparatus of any one of embodiments 1, 2, and 3, wherein the anesthesia transportation cart has a C_{H} of at least about 500 mm.

Embodiment 23. The portable anesthesia apparatus of any one of embodiments 1, 2, and 3, wherein the anesthesia transportation cart has a C_{BW} of not greater than about 800 mm.

Embodiment 24. The portable anesthesia apparatus of any one of embodiments 1, 2, and 3, wherein the anesthesia transportation cart has a C_{BW} of at least about 200 mm.

Embodiment 25. The portable anesthesia apparatus of any one of embodiments 1, 2, and 3, wherein the anesthesia transportation cart has a C_{BD} of not greater than about 1000 mm.

Embodiment 26. The portable anesthesia apparatus of any one of embodiments 1, 2, and 3, wherein the anesthesia transportation cart has a base depth of at least about 300 mm.

Embodiment 27. The portable anesthesia apparatus of embodiment 16, wherein the mounting post comprises at least one port, an O₂ tank mount and a N₂O tank mount.

Embodiment 28. The portable anesthesia apparatus of any one of embodiments 1, 2, and 3, wherein the anesthesia transportation cart comprises at least one power outlet.

Embodiment 29. The portable anesthesia apparatus of any one of embodiments 1, 2, and 3, wherein the anesthesia transportation cart comprises a CO₂ absorber mount.

Embodiment 30. The portable anesthesia apparatus of any one of embodiments 1, 2, and 3, wherein the anesthesia transportation cart comprises a lower equipment shelf.

Embodiment 31. The portable anesthesia apparatus of any one of embodiments 1, 2, and 3, wherein the anesthesia transportation cart comprises an anesthesia machine mount.

Embodiment 32. The portable anesthesia apparatus of any one of embodiments 1, 2, and 3, wherein the transportation cart comprises at least one gas port.

Embodiment 33. The portable anesthesia apparatus of any one of embodiments 1, 2, and 3, wherein the hospital-grade anesthesia machine is an FDA approved anesthesia machine, wherein the hospital-grade anesthesia machine is a three gas machine, wherein the hospital-grade anesthesia machine comprises a mechanical anti-hypoxic device, wherein the hospital-grade anesthesia machine comprises an air/N₂O interlock, wherein the hospital-grade anesthesia machine comprises ventilator options, wherein the hospital-grade anesthesia machine comprises a vaporizer mounting system, wherein the hospital-grade anesthesia machine comprises flush valves, wherein the hospital-grade anesthesia machine accommodates low flow operation.

Embodiment 34. The portable anesthesia apparatus of any one of embodiments 1, 2, and 3, wherein the hospital-grade CO₂ absorber comprises a high capacity CO₂ absorber/ventilator interface or wherein the CO₂ absorber is autoclavable or wherein the CO₂ absorber comprises Integrated spirometry and oxygen sensors or wherein the CO₂ absorber comprises changeable absorber material.

Embodiment 35. The portable anesthesia apparatus of any one of embodiments 1, 2, and 3, wherein the portable anesthesia apparatus further comprises an O₂ tank.

Embodiment 36. The portable anesthesia apparatus of embodiment 35, wherein the O₂ tank comprises an E-size O₂ tank.

Embodiment 37. The portable anesthesia apparatus of any one of embodiments 1, 2, and 3, wherein the portable anesthesia apparatus further comprises an N₂O tank.

Embodiment 38. The portable anesthesia apparatus of embodiment 37, wherein the N₂O tank comprises an E-size N₂O tank.

Embodiment 39. A portable anesthesia transportation cart comprising: a portable base; a mounting post extending up from the portable base; a CO₂ absorber mount attached to the mounting post; an equipment shelf attached to the mounting post; and an anesthesia machine mount attached to the equipment shelf; wherein the anesthesia transportation cart has a C_{FA} of not greater than about 1.0 m².

Embodiment 40. A portable anesthesia transportation cart comprising: a portable base; a mounting post extending up from the portable base; a CO₂ absorber mount attached to the mounting post; an equipment shelf attached to the mounting post; and an anesthesia machine mount attached to the equipment shelf, wherein the anesthesia transportation cart has an occupying volume of not greater than about 1.0 m³.

Embodiment 41. A portable anesthesia transportation cart comprising: a portable base; a mounting post extending up from the portable base; a CO₂ absorber mount attached to the mounting post; an equipment shelf attached to the mounting post; and an anesthesia machine mount attached to the equipment shelf, wherein the anesthesia transportation cart has a C_{H} of not greater than about 1500 mm, wherein the anesthesia transportation cart has a C_{BW} of not greater than about 800 mm, and wherein the anesthesia transportation cart has a C_{BD} of not greater than about 1000 mm.

Embodiment 42. The portable anesthesia transportation cart of any one of embodiments 39, 40, and 41, wherein the anesthesia transportation cart has a C_{FA} of not greater than about 0.5 m².

Embodiment 43. The portable anesthesia transportation cart of any one of embodiments 39, 40, and 41, wherein the anesthesia transportation cart has a C_{FA} of at least about 0.2 m².

Embodiment 44. The portable anesthesia transportation cart of any one of embodiments 39, 40, and 41, wherein the anesthesia transportation cart has an occupying volume of not greater than about 0.5 m³.

Embodiment 45. The portable anesthesia transportation cart of any one of embodiments 39, 40, and 41, wherein the anesthesia transportation cart has a C_{OV} of at least about 0.2 m³.

Embodiment 46. The portable anesthesia transportation cart of any one of embodiments 39, 40, and 41, wherein the anesthesia transportation cart has a C_{H} of not greater than about 1200 mm.

Embodiment 47. The portable anesthesia transportation cart of any one of embodiments 39, 40, and 41, wherein the anesthesia transportation cart has a C_{H} of at least about 600 mm.

Embodiment 48. The portable anesthesia transportation cart of any one of embodiments 39, 40, and 41, wherein the anesthesia transportation cart has a C_{BW} of not greater than about 500 mm.

Embodiment 49. The portable anesthesia transportation cart of any one of embodiments 39, 40, and 41, wherein the anesthesia transportation cart has a C_{BW} of at least about 300 mm.

Embodiment 50. The portable anesthesia transportation cart of any one of embodiments 39, 40, and 41, wherein the anesthesia transportation cart has a C_{BD} of not greater than about 700 mm.

Embodiment 51. The portable anesthesia transportation cart of any one of embodiments 39, 40, and 41, wherein the anesthesia transportation cart has a base depth of at least about 400 mm.

Embodiment 52. The portable anesthesia transportation cart of embodiment 51, wherein the mounting post comprises at least one port, an O₂ tank mount and a N₂O tank mount.

Embodiment 53. The portable anesthesia transportation cart of any one of embodiments 39, 40, and 41, wherein the anesthesia transportation cart comprises at least one power outlet.

Embodiment 54. The portable anesthesia transportation cart of any one of embodiments 39, 40, and 41, wherein the anesthesia transportation cart comprises a CO₂ absorber mount.

Embodiment 55. The portable anesthesia transportation cart of any one of embodiments 39, 40, and 41, wherein the anesthesia transportation cart comprises a lower equipment shelf.

Embodiment 56. The portable anesthesia transportation cart of any one of embodiments 39, 40, and 41, wherein the anesthesia transportation cart comprises an anesthesia machine mount.

Embodiment 57. The portable anesthesia transportation cart of any one of embodiments 39, 40, and 41, wherein the transportation cart comprises at least one gas port.

Embodiment 58. The portable anesthesia transportation cart of any one of embodiments 39, 40, and 41, wherein the hospital-grade anesthesia machine is an FDA approved anesthesia machine, wherein the hospital-grade anesthesia machine is a three gas machine, wherein the hospital-grade anesthesia machine comprises a mechanical anti-hypoxic device, wherein the hospital-grade anesthesia machine comprises an air/N₂O interlock, wherein the hospital-grade anesthesia machine comprises ventilator options, wherein the hospital-grade anesthesia machine comprises a vaporizer mounting system, wherein the hospital-grade anesthesia machine comprises flush valves, wherein the hospital-grade anesthesia machine accommodates low flow operation.

Embodiment 59. The portable anesthesia transportation cart of any one of embodiments 39, 40, and 41, wherein the hospital-grade CO₂ absorber comprises a high capacity CO₂ absorber/ventilator interface or wherein the CO₂ absorber is autoclavable or wherein the CO₂ absorber comprises Integrated spirometry and oxygen sensors or wherein the CO₂ absorber comprises changeable absorber material.

Embodiment 60. The portable anesthesia transportation cart of any one of embodiments 39, 40, and 41, wherein the portable anesthesia transportation cart further comprises an O₂ tank.

Embodiment 61. The portable anesthesia transportation cart of embodiment 60, wherein the O₂ tank comprises an E-size O₂ tank.

Embodiment 62. The portable anesthesia transportation cart of any one of embodiments 39, 40, and 41, wherein the portable anesthesia transportation cart further comprises an N₂O tank.

Embodiment 63. The portable anesthesia transportation cart of embodiment 62, wherein the N₂O tank comprises an E-size N₂O tank.

The present application represents a departure from the state of the art. Notably, the embodiments herein demonstrate improved and unexpected performance over conventional anesthesia apparatuses and transportation carts.

In the foregoing, reference to specific embodiments and the connections of certain components is illustrative. It will be appreciated that reference to components as being coupled or connected is intended to disclose either direct connection between said components or indirect connection through one or more intervening components as will be appreciated to carry out the methods as discussed herein. As such, the above-disclosed subject matter is to be considered illustrative, and not restrictive, and the appended claims are intended to cover all such modifications, enhancements, and other embodiments, which fall within the true scope of the present invention. Thus, to the maximum extent allowed by law, the scope of the present invention is to be determined by the broadest permissible interpretation of the following claims and their equivalents, and shall not be restricted or limited by the foregoing detailed description.

The Abstract of the Disclosure is provided to comply with Patent Law and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all features of any of the disclosed embodiments. Thus, the following claims are incorporated into the Detailed Description, with each claim standing on its own as defining separately claimed subject matter.

What is claimed is:

1. A portable anesthesia apparatus comprising:
an anesthesia transportation cart;
a hospital-grade anesthesia machine mounted on the anesthesia transportation cart; and
a hospital-grade CO₂ absorber mounted on the anesthesia transportation cart, wherein the portable anesthesia apparatus has an A_{FA} of not greater than about 1.0 m².

2. The portable anesthesia apparatus of claim 1, wherein the portable anesthesia apparatus has an A_{W} of not greater than about 210 kilograms.

3. The portable anesthesia apparatus of claim 1, wherein the portable anesthesia apparatus has an A_{FA} of at least about 0.1 m² and not greater than about 0.5 m².

4. The portable anesthesia apparatus of claim 1, wherein the portable apparatus has an A_{OV} of at least about 0.1 m³ and not greater than about 0.5 m³.

5. The portable anesthesia apparatus of claim 1, wherein the portable apparatus has an A_{H} of not greater than about 1200 mm.

6. The portable anesthesia apparatus of claim 1, wherein the portable anesthesia apparatus has an A_{H} of at least about 500 mm.

7. The portable anesthesia apparatus of claim 1, wherein the portable anesthesia apparatus has an A_{BW} width of not greater than about 550 mm.

8. The portable anesthesia apparatus of claim 1, wherein the portable anesthesia apparatus has an A_{BW} of at least about 200 mm.

9. The portable anesthesia apparatus of claim 1, wherein the portable anesthesia apparatus has an A_{BD} of not greater than about 700 mm.

10. The portable anesthesia apparatus of claim 1, wherein the portable anesthesia apparatus has an A_{BD} of at least about 300 mm.

11. The portable anesthesia apparatus of claim 1, wherein the anesthesia transportation cart comprises:
a portable base;
a mounting post extending up from the portable base;
a CO₂ absorber mount attached to the mounting post;
an equipment shelf attached to the mounting post; and
an anesthesia machine mount attached to the equipment shelf.

12. The portable anesthesia apparatus of claim 1, wherein the hospital-grade anesthesia machine is an FDA approved anesthesia machine.

13. The portable anesthesia apparatus of claim 1, wherein the hospital-grade CO₂ absorber comprises a high capacity CO₂ absorber/ventilator interface.

14. The portable anesthesia apparatus of claim 1, wherein the portable anesthesia apparatus further comprises an O₂ tank.

15. The portable anesthesia apparatus of claim 14, wherein the O₂ tank comprises an E-size O₂ tank.

16. The portable anesthesia apparatus of claim 1, wherein the portable anesthesia apparatus further comprises an N₂O tank.

17. The portable anesthesia apparatus of claim 16, wherein the N₂O tank comprises an E-size N₂O tank.

18. A portable anesthesia transportation cart comprising:
a portable base;
a mounting post extending up from the portable base;
a CO₂ absorber mount attached to the mounting post;
an equipment shelf attached to the mounting post; and
an anesthesia machine mount attached to the equipment shelf;
wherein the anesthesia transportation cart has a C_{FA} of not greater than about 1.0 m².

19. The portable anesthesia transportation cart of claim 18, wherein the anesthesia transportation cart has a C_{OV}, of not greater than about 1.0 m³.

20. The portable anesthesia transportation cart of claim 18, wherein the hospital-grade anesthesia machine is an FDA approved anesthesia machine.
